(19) United States
(12) Patent Application Publication
(10) Pub. No.: US 2026/0287473 A1
(43) Pub. Date: Sep. 24, 2026

(54) DEVICE FOR TAKING SOIL SAMPLES

(71) Applicant: FNV IP B.V., Leidschendam (NL)

(72) Inventors: Peter Nicolaas LOOIJEN, Leidschendam (NL); Verónica SUAREZ MENDEZ, Leidschendam (NL); William Ray WOOD, Leidschendam (NL)

(73) Assignee: FNV IP B.V., Leidschendam (NL)

(21) Appl. No.: 19/473,424

(22) PCT Filed: Apr. 29, 2024
(86) PCT No.: PCT/EP2024/061719

§ 371 Date: Oct. 7, 2025

(30) Foreign Application Priority Data
May 2, 2023 (NL) 2034745

(51) Int. Cl. G01N 1/08; G01N 33/24

(52) U.S. Cl. CPC G01N 1/08; G01N 33/24

(57) ABSTRACT

Sampling device and method for determining void ratio, comprising a housing, arranged to be coupled to a drive system, the housing defining a distal section and a proximal section, a sampling unit provided in a space defined by the housing, the sampling unit comprising a volumetrically calibrated sampling chamber, which can be moved between a closed position and an open position, and an actuation mechanism, and a method of retrieving a soil sample to determine a void ratio of said soil sample, a kit of parts for assembling a sampling device, and the use of a sampling device. Unlocking insights from Geo-Data, the present invention further relates to improvements in sustainability and environmental developments: together we create a safe and liveable world.

### FIELD OF THE INVENTION

The present disclosure generally relates to a sampling device for retrieving a soil sample to determine a void ratio of said soil sample, and more specifically to a method of retrieving a soil sample to determine a void ratio of said soil sample, to a kit of parts for assembling a sampling device and to the use of a sampling device. Unlocking insights from Geo-Data, the present invention further relates to improvements in sustainability and environmental developments: together we create a safe and liveable world.

### BACKGROUND OF THE INVENTION

There is a general and ongoing need to improve the quality and efficiency of in-situ soil sampling. In particular, there is a growing need to ensure retention of the constituents of soil sample for the determination of void ratios and degree of saturation of soil samples.

Subsurface testing is generally performed to determine the characteristics of the soil at certain depths. Such subsurface characteristics may be soil type, density, moisture content, shear modulus, and the like, which may be used in foundation planning and/or management or in determination of soil stability for risk assessments. Such soil characteristics may play a vital role in e.g., infrastructure projects, but may also be used to map soil characteristics for different purposes such as environmental projects, coastal resilience projects, or dam integrity projects.

One example of subsurface soil characteristics is the void ratio, which is a measure of of the amount of fluid (i.e., gaseous or liquid) volume in a soil, in between solid particles. The determination of the void ratio and the associated degree of saturation (for partially saturated soils) of the soil is of particular importance in the determination of stability of soils that might be susceptible to static liquefaction. Static liquefaction is the sudden loss of strength when loose soil, typically granular material such as sand or silty sand, is loaded and cannot drain. As an example, tailings facilities are formed by continuous hydrodynamic deposit of soil. Tailings are a very loose soil and have a relatively low in-situ density as a result. The tailings generally contract on the application of shear force leading to a high tendency for brittle failure and liquefaction leading to damaging mud flows. Similar situations can arise, e.g., in storage facilities for materials generated by maintenance dredging of navigation channels, for ash deposits generated by the power generation industry and in some naturally occurring soils.

Since such brittle failure is sudden, it has a high potential of dangerous consequences. To predict whether brittle failure is likely to occur, a measure of the void ratio in relation to the critical state void ratio at the same mean effective stress is used. The critical state (or critical void ratio) line of a soil is the locus of void ratio-effective stress conditions achieved after shearing a soil to large displacement and after all net void ratio changes and effective stress changes are complete. Generally, if the in-situ void ratio is greater than that for the critical state for the same mean effective stress (analogous to depth), the material will be susceptible to static liquefaction increasing the risk and consequence of failure of the retaining structure and the tailings impounded behind it.

For the reliable assessment of the likelihood of static liquefaction within a tailings facility, an accurate determination of the in-situ void ratio is important.

The current approach to determining liquefaction susceptibility uses the resistance of a cone penetration test (CPT). The difference between in-situ void ratio and the corresponding void ratio at the critical state (known as the ‘state parameter’) is correlated with the cone resistance to determine this property as a function of cone resistance in CPT tests. However, the use of cone resistance to determine the ‘state parameter’ has proven to lack accuracy and consistency, leading to qualitatively poor predictions about the stability of the soil.

A known solution to these problems is to create a borehole to take samples at depth. However, to drill a borehole prior to testing the soil increases the sampling time and cost. Further, in some cases, the boreholes may be unstable, leading to their collapse prior to sampling. The creation of boreholes prior to sampling may also consolidate the soil through vibrations during drilling, leading to a lower measured void ratio. The action of conventional sampling using long, thin-walled tubes pushed into soil at the base of the borehole may lead to densification of these very loose soils due to the build-up of friction on the inner wall of the sampler. Also, these loose materials are notoriously difficult to retain in the sample tube during recovery to the surface from the base of the borehole, meaning that some of the constituents of the sample can be lost resulting in an inaccurate assessment of in-situ voids ratio. As a result, the void ratio seems lower than it actually is, and a soil sample may be deemed stable while the in-situ void ratio is much higher and the soil is actually unstable.

The known state of the art for in-situ void ratio sampling thus lacks a solution for retrieving samples having in-situ representations of its constituents. There is thus a need for an improved device and method for determining in-situ void ratio.

### BRIEF SUMMARY OF THE INVENTION

According to one aspect of the present disclosure, there is provided a sampling device for retrieving a soil sample from a target soil to determine a void ratio of said soil sample. In an example the sampling device comprises a housing, a sampling unit having a volumetrically calibrated sampling chamber, and an actuation mechanism, arranged to move the sampling unit between a closed position and an open position. Hereinafter, a reference to the open position of the sampling device, or the sampler, means that the sampling unit is provided in the open position. Similarly, a reference to the closed position of the sampling device, or the sampler, means that the sampling unit is provided in the closed position.

In an example, the housing is arranged to be coupled to a drive system. The housing may define a distal section and a proximal section. The distal section of the housing may have an open end arranged to receive soil as the sampling device is driven into the target soil.

In an example, the sampling unit may be provided in a space defined by the housing, preferably in a space defined by the distal section and the proximal section of the housing. The space may extend into the housing from the open end of the distal section. The sampling unit may comprise a volumetrically calibrated sampling chamber. The volumetrically calibrated sampling chamber may extend at least partly through the sampling unit, such that the sampling chamber defines at least one opening in the sampling unit.

In an advantageous example embodiment, the sampling unit may have a substantially spherical shape. The sampling unit may be provided in the form of a ball-valve. The outer surface of the sampling unit may be substantially spherical. In an embodiment, the outer surface of the sampling unit may have a substantially circular cross-section. The sampling unit may have flattened sides. The actuation mechanism may be attached to the sampling unit on those flattened sides. The sampling unit may be provided directly in the housing or in a container. In an advantageous embodiment, the outer surface of the sampling unit conforms to the inner surface of the housing or the inner surface of the container.

In an example, the sampling unit may be rotatably connected to the housing such that it can rotate between a closed position and an open position. In an example, in the closed position, the opening defined by the volumetrically calibrated sampling chamber faces away from the open end of the distal section of the housing. In the closed position, the at least one opening in the sampling unit is sealed. In the closed position, the at least one opening in the sampling unit may be sealed by a container, the container being provided in the housing, and around the sampling unit.

Advantageously, by providing a container around the sampling unit, the retrieval of the sampling unit from the sampling device in the field is simplified. Since the constituents in the volumetrically calibrated sampling chamber of the sampling unit have to be retained for an accurate assessment of the void ratio, it is beneficial if the sampling chamber is sealed until it arrives at a testing facility. To accomplish this, the sampling unit may be provided in a container, which is held in the housing and seals the sampling unit with the volumetrically calibrated sampling chamber. The container is then removed from the housing and brought to a testing facility without compromising the soil constituents in the sampling chamber.

In an alternative example embodiment, the at least one opening in the sampling unit may be sealed directly by the housing. In such an embodiment, the sampling unit is in direct contact with the housing of the sampling device.

In such an embodiment, the sampling unit is provided directly in the housing such that, to retain sample integrity, the entire housing is removed from the sampling device and brought to the testing facility. Such an arrangement will require more material in the field, since a housing is used for every soil sample. However, it may increase simplicity in the design of the sampling device as a whole.

By having a container around the sampling unit, the sampling unit and container can be removed from the housing, a new container and new sampling unit may be provided in the housing, and the device can take another sample.

In an advantageous embodiment, the container comprises two sections, which may be disengaged to allow access to the sampling unit. In an embodiment, the container has a split down the middle in a vertical direction such that it comprises two substantially symmetrical halves. In an alternative embodiment, the container may comprise two halves which are split along a horizontal and/or a diagonal line. In an embodiment, the two halves of the container may have a hinged attachment.

In an example, in the open position, the opening defined by the volumetrically calibrated sampling chamber faces the open end of the distal section of the housing. In an example, the actuation mechanism is arranged to rotate the sampling unit between the closed position and the open position.

The sampling device according to the present disclosure allows the retrieval of a sample, without disturbing the division of its constituents. That is, the sampling device is able to retrieve a sample in which the relative amounts of fluid (gaseous or liquid) and solid materials are maintained. It is noted that an unsaturated sample, which is above the water table, will contain both gaseous and liquid constituents, which both must be retained. Such retention of all constituents allows determination of the degree of saturation, another important soil mechanics parameter. The present invention allows the retrieval of samples in which all constituents, also gaseous and liquid, are maintained.

As a result, the sample can be analysed after retrieval to determine the in-situ constituent composition. As the sampling unit is provided in a rotatable fashion within the housing, and the opening of the sampling chamber is sealed in the closed position, the constituents of the soil sample are retained as they are found in-situ. As such, the soil sample can be retrieved, even in loose soil types, while maintaining the ratio of the constituents. The maintenance of constituents in the context of this disclosure shall hereinafter be referred to as a sample being undisturbed.

The retrieval of undisturbed soil samples allows the void ratio to be determined much more accurately, and without undue compression of the soil as the sample is taken. This advantageously allows for a more accurate determination of the static liquefaction potential of the soil, thus improving calculations relating to the stability of a soil type in, e.g., tailings dams. It shall be understood that the sampling device may be used to retrieve soil samples from any type of soil, in which the retrieval of an undisturbed soil sample is required.

In an example, the housing of the sampling device may comprise a cylindrical outer shape, such that its outer diameter is substantially equal to or greater than the outer diameter of the drive system. The distal section of the housing comprises an open end such that soil can enter the sampling device as it is advanced into the soil where a sample is to be taken. The open end of the distal section of the housing is arranged such that it receives soil as the sampling device is driven into the target soil. In an example, the open end of the distal section of the housing may coincide with the opening defined by the volumetrically calibrated sampling chamber, when the sampling unit is rotated to the open position.

Advantageously, the open end of the distal section of the housing is positioned away from the opening defined by the volumetrically calibrated sampling chamber such that the open end of the distal section is the distal most part of the sampling device. As the sampling device is driven into the soil, the housing may partly receive soil through the open end of the distal section. In an example, the open end of the distal section receives soil as the sampling device is driven into the soil, but the soil does not enter the sampling unit. The distal section of the housing may be at least partly closed off by the sampling unit in its closed position. The position where the sampling unit closes the distal section of the housing may coincide with the open end of the distal section of the housing. Alternatively, the sampling unit may close the distal section of the housing at a position further inward from the open end of the distal section of the housing.

Once a required depth is achieved, the sampling unit may be rotated to the open position, thereby also allowing soil to be received through the distal section of the housing, into the volumetrically calibrated sampling chamber. In an advantageous example, the proximal section of the housing of the sampling device is arranged to be coupled to the drive system. In an example embodiment, the proximal section of the housing of the sampling device comprises a threaded section arranged to be connected to a threaded section of the drive system.

A drive system in the context of the present invention may be any device that is able to drive the sampling device into the soil. Such a device generally is an elongated in-situ testing device, which is able to bring the sampling device to the required depth for sampling the soil. In an example embodiment, the drive system may be a coiled penetrometer system, comprising a coiled penetrometer rod.

In another example, the drive system may be a conventional penetrometer system, having plurality of stacked rods to drive the sampling device into the soil. In an example embodiment, a borehole may have already been created, and the drive system is only required to drive the sampling device into the soil at the bottom of the borehole. In an advantageous example, the drive system is arranged such that the drive system can drive the sampling device through the soil to the required depth, without the need for a borehole.

In an example embodiment, the distal section of the housing is detachably connected to the proximal section.

The distal section of the housing may be connected to the proximal section by any detachable connection, such as a threaded connection. In other example embodiments, the distal section and the proximal section may be clamped together, or provided with clips, or using bolts or the like to facilitate the connection.

Advantageously, by providing a detachable connection between the distal section and the proximal section of the housing, the sampling unit may be retrieved from the housing. In an advantageous example embodiment, the sampling unit is contained between the distal section of the housing and the proximal section of the housing. In such an embodiment, the sampling unit can only be removed from the housing by detaching the distal section from the proximal section of the housing.

In an example embodiment, the open end of the distal section of the housing axially aligns with the at least one opening in the sampling unit, defined by the volumetrically calibrated sampling chamber, when the sampling unit is in the open position.

By having the open end of the distal section of the housing align with the opening in the sampling unit, which is defined by the sampling chamber, less friction is induced as the soil enters the sampling chamber via the open end of the distal section of the housing. Advantageously, this leaves the soil less disturbed (i.e., compressed or otherwise influenced) as it is brought into the sampling chamber.

The alignment of the open end of the distal section of the housing with the at least one opening in the sampling unit, defined by the volumetrically calibrated sampling chamber, is axial. That is, volumetrically calibrated sampling chamber extends into the sampling unit such that the opening defined in the sampling unit aligns with the open end of the distal section of the housing. That is, the space defined by the housing, extending into the housing from the open end of the distal section, aligns with the opening in the sampling unit such that no change in circumference is present, when moving from the space in the distal end of the housing, towards the volumetrically calibrated sampling unit.

In an example embodiment the volumetrically calibrated sampling chamber defines a through-hole in the sampling unit. In such an embodiment, the sampling unit comprises two openings, the opening and a further opening, and the volumetrically calibrated sampling chamber is limited by the two openings of the sampling unit. In an advantageous embodiment, the volumetrically calibrated sampling chamber defines a cylindrical shape. Since the drive system generally defines a cylindrical outer shape, having a cylindrical sampling chamber reduces the total influence on the target soil as the soil is advanced into the sampling device.

Advantageously, the cross-sectional shape of the volumetrically calibrated sampling chamber is constant over a length of the volumetrically calibrated sampling chamber, extending inward from the opening of the sampling unit. In an advantageous embodiment, the volumetrically calibrated sampling chamber extends between the opening of the sampling unit and the further opening of the sampling unit and has a constant cross-sectional shape over the length of the volumetrically calibrated sampling chamber.

In an advantageous example embodiment, the cross-sectional shape of the volumetrically calibrated sampling chamber corresponds to the shape of the open end of the distal end of the housing, and similarly to the space in the distal end of the housing, between the open end of the distal end of the housing and the sampling unit. This arrangement reduces variations in the shape of the spaces through which the soil must travel during collection of a soil sample.

In an advantageous embodiment, the proximal section of the housing comprises a discharge volume, such that the volumetrically calibrated sampling chamber is positioned between the discharge volume and the open end of the distal section of the housing. This advantageously allows soil to be driven through the through-hole in the sampling unit and received in the discharge volume of the proximal section of the housing.

In an advantageous embodiment, the discharge volume is filled with a liquid, preferably water. Advantageously, the discharge volume is filled with a gas or a liquid having a low coefficient of compression. That is, the reduction of the total volume under a given pressure is small. By filling the discharge volume with a liquid, or a gas with a low coefficient of compression, the increase in pressure on the sampling device leads to a lower decrease of volume, such that the sampling unit is not wedged against the housing or against the container.

The soil just below the sampling device is likely compressed due to the penetration of the soil by the sampling device. If a sample is then taken from that compressed soil, the measured void ratio differs from the actual in-situ void ratio. By allowing soil to pass through the volumetrically calibrated sampling chamber, the compressed soil is advanced into the discharge volume, and the soil in the volumetrically calibrated sampling chamber is less compressed. As the sampling unit is then rotated from the open to the closed position, the sample retained in the volumetrically calibrated sampling chamber has a void ratio which more closely resembles the in-situ void ratio.

In an example embodiment, the discharge volume of the proximal section of the housing comprises a release valve, arranged to allow air to escape from the discharge volume as soil enters into the discharge volume.

In an advantageous embodiment, the cross-sectional shape of the discharge volume, perpendicular to an axial direction of the sampling device, is larger than the cross-sectional shape of the volumetrically calibrated sampling chamber. As a result, once the compressed soil is driven through the open end of the distal section of the housing, and through the through-hole of the sampling unit, defined by the volumetrically calibrated sampling chamber, the inner diameter of the discharge volume reduces the pressure on the soil. This reduces the required length of the discharge volume such that the retrieval of an undisturbed soil sample is improved.

In an embodiment, the sampling device further comprises a moving plug provided in the open end of the distal section of the housing. In this embodiment, the moving plug is arranged to move through the through-hole defined in the sampling unit by the volumetrically calibrated sampling chamber. As the sampling device is entered into the soil, the sampling unit is provided in the closed position, such no matter can move from the open end of the distal section of the housing to the discharge volume above the sampling unit. As a result, as the sampling device is driven into the soil, the moving plug provides a barrier for soil to enter the open end of the distal section of the housing.

Once the sampling device reaches target depth, the actuation mechanism turns the sampling unit to the open position, such that the through-hole defined by the volumetrically calibrated sampling chamber connects the open end of the distal section of the housing with the discharge volume. As the sampling device is then advanced into the soil, the moving plug is pushed through the sampling unit into the discharge volume and fresh soil is entered into the sampling device, and into the volumetrically calibrated sampling chamber.

Advantageously, the use of such a moving plug prevents soil from near surface from entering the sampling device as it is driven into the soil. As such, it reduces intermixing of materials from different depths in the open end of the distal section of the housing.

Further, it may be possible, when pushing from the surface through a coarser material, that such material could become packed and locked in the distal section of the housing. If that happens, even if the sampling unit is rotated into the open position, the internal friction in the distal section of the housing alone would be sufficient to cause the sampling device to penetrate as if closed. This would prevent the material of interest entering the volumetrically calibrated sampling chamber, thus negating the purpose of the exercise.

In an advantageous embodiment, the moving plug comprises a cone-shaped distal tip. In an advantageous embodiment the shape of the tip of moving plug conforms to the shape of the distal section of the housing, such that friction is reduced as the sampling device is driven into the soil. In an advantageous embodiment, the moving plug comprises at least one low-friction seal, allowing it to slide through the housing, and through the volumetrically calibrated sampling chamber, and into the discharge volume with reduced force. In an embodiment, the top of the moving plug comprises a low-friction material, such as Teflon. Advantageously, this reduces the force required to rotate the sampling unit, even if the moving plug presses against the sampling unit as it is driven into the soil.

In an advantageous embodiment, the moving plug comprises a depth sensor, arranged to measure a depth of the moving plug. In an example embodiment, the moving plug comprises a pore pressure sensor, arranges to measure porewater pressure. This allows to refine future sampling efforts and to allow a measurement of hydro geological conditions before sampling commences.

In an example embodiment, an outer surface of the sampling unit defines a circular periphery at a cross-section perpendicular to an axis of rotation of the sampling unit.

By having a circular periphery, the sampling unit may rotate without losing contact with the inner surface of the space defined by the housing. As a result, the soil sample is maintained in the volumetrically calibrated sampling volume as the sampling unit is rotated from the open position to the closed position.

In an example embodiment, the actuation mechanism comprises at least one longitudinally extending actuation rod, and wherein the sampling unit is coupled to the actuation rod, the actuation rod extending through the housing to the sampling unit.

In an additional or alternative example embodiment, the actuation mechanism comprises at least one of a gear, a belt, and a chain. The rotation of the sampling unit may be achieved through a selection of gears, which actuate the sampling unit. The sampling unit may further be actuated through a belt or a chain, which is driven such that the sampling unit can be rotated between the closed and open positions.

The actuation mechanism may utilise electrical or mechanically stored energy, such as springs, such as disc springs, helical springs, or the like, or using hydraulics, such as gas springs, hydraulic springs, or active hydraulic systems using pumped hydraulic fluids (gaseous or liquid). The actuation mechanism may comprise a hydraulic system which utilises fluid pressure to move the sampling unit between the closed and the open position or vice versa.

In an example embodiment, the at least one actuation rod is coupled to the sampling unit via at least one pin, the pin extending into the rod and into the sampling unit in a position away from an axis of rotation of the sampling unit, such that an axial force exerted to the rod create a rotational moment on the sampling unit around the axis of rotation.

In an example embodiment, the actuation mechanism comprises a hydraulic drive system, such that the sampling unit may be moved between an open and a closed position by the increase or decrease of a hydraulic pressure.

In an example embodiment, the actuation mechanism comprises an electric drive system, comprising an electric motor arranged to drive rotation of the sampling unit between a closed and an opened position.

In an example embodiment, the actuation mechanism comprises a cylindrical piston provided within the proximal section of the housing, the cylindrical piston being coupled to at least one actuation rod to drive the rotation of the sampling unit.

In an example embodiment, the actuation mechanism comprises a biasing element to bias the sampling unit in the closed position or in the opened position.

Having a biasing element to bias the sampling unit prevents the need for bi-directional actuation of the sampling unit. Advantageously, by biasing the sampling unit in e.g., the closed position, the sampling device may be driven into the soil in a passive state, and the actuation device may drive the sampling unit to overcome the biasing element and open the sampling unit as the soil sample is taken. After the sample is taken, the actuation mechanism releases, and the biasing element brings the sampling unit back in closed position to seal the volumetrically calibrated sampling chamber.

In an example embodiment, the biasing element is a helical spring, arranged within the proximal section of the housing, and wherein the actuation mechanism comprises a cylindrical piston provided within the proximal section of the housing, wherein the biasing element is arranged to bias the cylindrical piston away from the sampling unit.

In an advantageous embodiment, the biasing element is a disc spring, having a plurality of coned rings, which are stacked. Such disc springs are also known as Belleville washers, conical spring washers, or cupped spring washers. Such a biasing element is able to withstand higher pressures and as such is particularly suited for use in the sampling device.

In an example embodiment, the housing comprises a metal, such as at least one of stainless steel, brass, carbon steel, copper alloys, and other high strength alloys.

Advantageously, the housing comprises stainless steel and brass. Stainless steel to stainless steel threaded connections have a tendency to gall during assembly or disassembly where small amounts of swarf in the threads can pressure weld the threads together, making them impossible to separate. Therefore, advantageously, the mating threaded surfaces may be brass and stainless steel. In such an advantageous embodiment, one of the proximal or the distal section of the housing comprises stainless steel, while the other comprises brass. The connection between the two housing sections will then be less susceptible to galling.

In an advantageous embodiment, the sampling unit, particularly the inner surface of the sampling unit defining the volumetrically calibrated sampling chamber, comprises a material with a low surface roughness, such as a metal. In an advantageous embodiment, such material may be a polished metal, such as highly polished brass to further reduce the internal friction. Additionally, or alternatively, the inner surface of the sampling unit defining the volumetrically calibrated sampling chamber comprises chrome plating to further reduce friction. The reduction of internal friction limits soil compression during the retrieval of an undisturbed sample.

In an advantageous embodiment the distal section of the housing comprises a channel through which a lubrication gel can be introduced to the inner surface of the distal end of the housing, defining the space in the housing. As a result, the friction between inner walls of the distal section of the housing and the soil moving through the housing is reduced. The friction in the volumetrically calibrated sampling chamber will then also be reduced as a result.

In an example embodiment, the housing comprises a ceramic, a composite material such as carbon fibre, or a metal, advantageously a metal taken from the group of steel, stainless steel, titanium, aluminium, brass, copper, or the like.

In an example embodiment, the volumetrically calibrated sampling chamber defines a volume of between about 10 cm³ and 200 cm³ , advantageously between about 30 cm³ and 150 cm³, more advantageously between about 50 cm³ and 120cm³, still more advantageously between about 60 cm³ and 90 cm³.

The volume defined in the volumetrically calibrated sampling chamber may be an optimized volume with relation to an outer diameter and an inner diameter of the housing of the sampling device.

In an example embodiment, the housing of the sampling device defines a maximum outer diameter of between about 20 mm and 300 mm, advantageously of between about 40 mm and 200 mm, more advantageously of between about 50 mm and 120 mm, still more advantageously of between about 60 mm and 90 mm.

In an example embodiment, the volumetrically calibrated sampling chamber has a diameter of between about 10 mm, and 100 mm, advantageously of between about 15 mm and 80 mm, more advantageously of between about 20 mm and 60 mm, still more advantageously of between 30 mm and 50 mm.

According to an aspect of the present disclosure, there is provided a method of retrieving a soil sample to determine a void ratio of said soil sample. In an example embodiment, the method comprises providing a sampling device according to any of the embodiments described in this disclosure. In an example embodiment, the method further comprises driving the sampling device into a target soil to a pre-determined target depth. The method may further comprise rotating the sampling unit from the open position into the closed position to seal a retrieved sample in the volumetrically calibrated sampling chamber. The method may further comprise retrieving the sampling device from the target soil.

In an example embodiment, the method further comprises removing the sampling unit from the housing after the sampling device is retrieved from the target soil.

In an example embodiment, the sampling unit is in a closed position as the sampling device is driven into the target soil. In an example embodiment, the method further comprises, once the sampling device is at the target depth, rotating the sampling unit from the closed position into the open position. In an example embodiment, the method may further comprise driving the sampling device further into the target soil for a pre-determined distance, prior to rotating the sampling unit back into the closed position.

By driving the sampling device into the soil while having the sampling unit in a closed position, soil does not already enter the volumetrically calibrated sampling chamber. As a result, the sampling device can be used at depth without the need to first create a borehole. This increases efficiency of the sampling method. In addition, it improves the quality of the taken samples as the creation of a borehole prior to sampling may influence the target soil due to e.g., vibration and/or compression.

According to an aspect of the present disclosure, there is provided a kit of parts for assembling a sampling device according to any of the embodiments described in this disclosure. In an example embodiment, the kit of parts comprises a housing, arranged to be coupled to a drive system, the housing defining a distal section and a proximal section, the distal section of the housing having an open end arranged to receive soil as the sampling device is driven into the soil.

In an example embodiment, the kit of parts comprises a sampling unit arranged to be provided in a space defined by a housing, preferably defined by the distal section and the proximal section of the housing. In an example, the space extending into the housing from the open end of the distal section. In an example, the sampling unit comprises a volumetrically calibrated sampling chamber. In an example, the volumetrically calibrated sampling chamber extends at least partly through the sampling unit, such that the sampling chamber defines at least one opening in the sampling unit.

In an embodiment, the sampling unit is arranged to be rotatably connected to the housing such that it can rotate between a closed position and an open position. In an example embodiment, in the closed position, the opening defined by the volumetrically calibrated sampling chamber faces away from the open end of the distal section of the housing. In an example embodiment, in the closed position, the at least one opening in the sampling unit is sealed. In the closed position, the at least one opening in the sampling unit may be sealed by a container, the container being provided in the housing, and around the sampling unit.

In an alternative example embodiment, the at least one opening in the sampling unit may be sealed directly by the housing. In such an embodiment, the sampling unit is in direct contact with the housing of the sampling device.

In an example embodiment, in the open position, the opening defined by the volumetrically calibrated sampling chamber faces the open end of the distal section of the housing.

In an embodiment, the kit of parts comprises an actuation mechanism, arranged to rotate the sampling unit between the closed position and the open position.

According to an aspect of the present disclosure there is provided the use of a sampling device according to any of the embodiments in this disclosure, for the retrieval of a sample for the determination of an in-situ void ratio of the sample.

Various example embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Specific details are described to provide a thorough understanding of the disclosure. However, in certain instances, well-known or conventional details are not described in order to avoid obscuring the description. A reference to an embodiment in the present disclosure can be a reference to the same embodiment or any other embodiment. Such references thus relate to at least one of the embodiments herein.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by various embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In various cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Without intent to limit the scope of the disclosure, examples of instruments, apparatus, methods, and their related results according to the embodiments of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are therefore not to be considered to be limiting of its scope, the principles herein are described and explained with additional specificity and detail through the use of the accompanying drawings in which:

FIG. 1 shows a cross-sectional view of the sampling device according to one example embodiment of the invention, in closed position;

FIG. 2 shows a cross-sectional view of the sampling device according to one example embodiment of the invention in open position;

FIG. 3 shows a three-dimensional cut-away view of the sampling device according to one example embodiment of the invention, in closed position;

FIG. 4 shows a three-dimensional cut-away view of the sampling device according to one example embodiment of the invention, in open position;

FIG. 5 shows a blow-out view of the sampling device, showing the sampling unit, the container, and part of the actuation mechanism according to an example embodiment of the invention;

FIG. 6 shows a three-dimensional cut-away view of the sampling device, showing the sampling unit, provided within the container, according to an example embodiment of the invention;

FIG. 7 shows a three-dimensional view of the sampling device, showing the sampling unit, the moving plug, and at least a part of the actuation mechanism, according to an example embodiment of the invention;

FIG. 8 shows a side view of the sampling device attached to a drive system provided in a truck, according to an example embodiment of the invention; and FIG. 9 shows a schematic diagram showing a method of retrieving a soil sample to determine a void ratio of said soil sample according to an example embodiment of the invention.

### DESCRIPTION OF ILLUSTRATIVE EMBODIMENTS

The following is a description of certain embodiments of the invention, given by way of example only and with reference to the drawings.

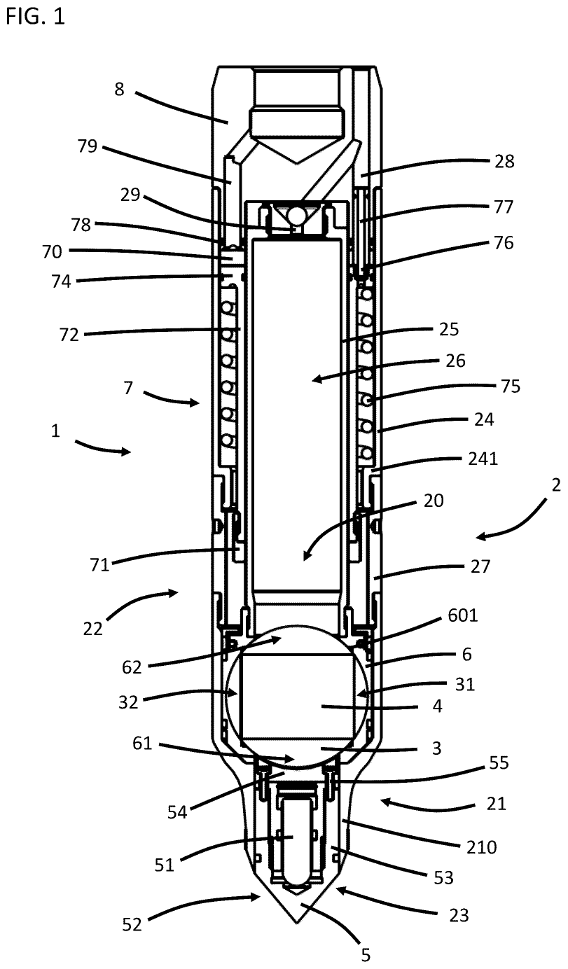

Referring to FIG. 1, a cross-sectional view of the sampling device 1 according to one example embodiment of the invention is shown. The sampling device 1 comprises a housing 2, arranged to be coupled to a drive system, not shown. The housing of the shown embodiment comprises a distal section 21 and a proximal section 22. The distal section 21 of the housing 2 comprises an open end 23 arranged to receive soil as the sampling device 1 is driven into the target soil. The distal section 21 of the housing 2 comprises a cutting shoe 210 in the shown embodiment. In the shown embodiment the distal section 21 of the housing 2 only comprises the cutting shoe 210. In other embodiments, the distal section 21 of the housing 2 may comprise further parts. It is noted that in the shown embodiment, the open end 23 of the distal section 21 of the housing is filled with a moving plug 5. However, the distal section 21 of the housing 2 still comprises an open end 23, even if it is filled with soil and/or a moving plug 5 or the like.

The sampling device 1 further comprises a sampling unit 3 provided in a space 20 extending into the housing 2 from the open end 23 of the distal section 21 of the housing 2. The sampling unit 3 comprises a volumetrically calibrated sampling chamber 4. The volumetrically calibrated sampling chamber 4, also referred to as the sampling chamber 4, extends at least partly through the sampling unit 3, such that the sampling chamber 4 defines at least one opening 31 in the sampling unit 3. In the shown embodiment, the sampling chamber 4 defines a first opening 31 and a second opening 32 opposite the first opening 31 such that a through-hole is defined by the sampling chamber 4 through the sampling unit 3.

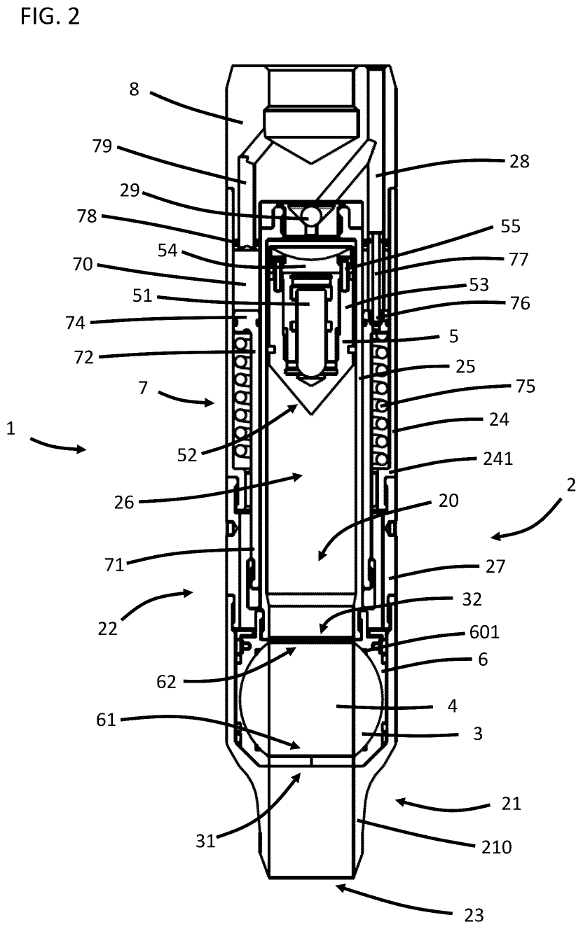

The sampling unit 3 is rotatably connected to the housing 2 such that it can rotate between the shown closed position and an open position, shown in FIG. 2. In the closed position, the first opening 31 and second opening 32 defined by the volumetrically calibrated sampling chamber 4 in the sampling unit 3 face away from the open end 23 of the distal section 21 of the housing 2, such that the first opening 31 and the second opening 32 in the sampling unit 3 are sealed. As can be seen in the shown embodiment, the first opening 31 and the second opening 32 are blocked by a container 6 which is provided in the housing. In other embodiments, the openings 31, 32 may be blocked by the housing 2 or by another element in the sampling device 1. The container 6 comprises an open bottom 61 and an open top 62, such that a sample can move through the container 6.

In the shown embodiment, a sampling unit seal 601 is provided in the container 6, such that it extends around the circumference of the sampling unit 3. The sampling unit seal 601 may also be provided in the housing 2 if the container 6 is not used in that embodiment. The sampling unit seal 601 advantageously comprises a low friction material, and is designed such that it cuts through the soil as the sampling unit 3 rotates between the closed and open positions.

The interior shape of the container 6 in the shown example embodiment has a substantially spherical shape. The outer shape of the sampling unit 3 is substantially spherical and conforms to the interior shape of the container. As such, the sampling unit 3 is able to rotate in the container 6. In the shown embodiment, the sampling unit 3 is rotated such that the opening 31 faces away from the open end 23 of the distal section 21 of the housing 2. As it is rotated to the open position, shown in FIG. 2, the volumetrically calibrated sampling chamber 4 aligns with the space 20 defined by the housing 2, such that a soil sample can enter through the open end 23 to the sampling chamber 4.

In the shown embodiment, the moving plug 5, provided in the open end 23 of the distal end 21 of the housing 2, comprises a depth sensor 51, arranged to measure the depth of the sampling device 1 as it is progressed into the soil to take a sample. The moving plug 5 further comprises a cone-shaped distal tip 52, arranged to reduce friction as the sampling device 1 is driven into the soil. The depth sensor 51 is provided in the interior of the moving plug 5. Other sensors, such as pore pressure sensors, temperature sensors, electrical conductivity sensors, or the like may similarly be provided in the moving plug 5.

The moving plug 5 comprises a plug body 53 and a top 54 which is connected to the moving plug body 53 by attachment units 55 such as screws, bolts, rivets, or the like. The top 54 of the moving plug 5 advantageously comprises a low-friction material, such as PTFE (polytetrafluoroethylene), also known as Teflon, polyimide, PEEK (polyether ether ketone), PPS (polyphenylene sulphide), Nylon, Acetal, or polyester. The top 54 of the moving plug 5 may also comprise low-friction metals, such as graphite, phosphor-bronze, cast iron, acrylic, copper, brass, tungsten carbide, aluminium, or the like.

Due to the pressure required to drive the sampling device 1 into the soil, there will be a compressive force between the top 54 of the moving plug 5 and the sampling unit 3. By providing the top 54 of the moving plug 5 with a low-friction material, the force required to rotate the sampling unit 3 between the closed to the open position, is reduced.

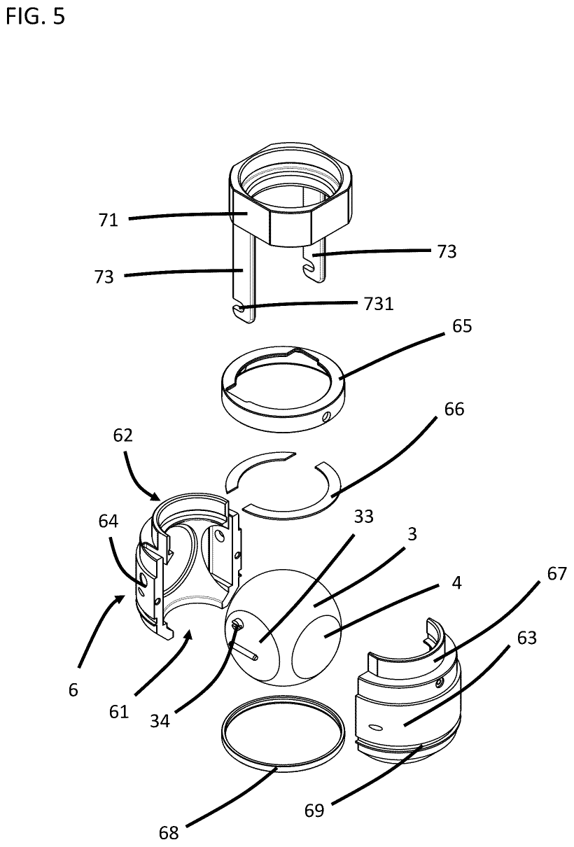

The sampling device 1 further comprises an actuation mechanism 7. The actuation mechanism comprises at least one longitudinally extending actuation rod 73, shown in FIG. 5 but not shown in the cross-sectional view of FIG. 1 or 2. The longitudinally extending actuation rod 73, also referred to as the actuation rod 73, is attached to drive ring 71. In the example embodiment of FIG. 5, the drive ring 71 is integrally formed with two opposing actuation rods 73. The actuation mechanism is provided in the proximal section 22 of the housing 2. The proximal section 22 of the housing 2 comprises an outer part 24, an inner part 25, and a lower part 27. The lower part 27 and the outer part 24 of the housing 2 are provided around the inner part 25 of the housing, such that the inner part 25 of the housing 2 extends through the combination of the outer part 24 and the lower art 27 of the housing. The drive ring 71 is provided around the inner part 25 of the housing 2, which defines the discharge volume 26 above the sampling unit 3 and within the outer part 24 of the housing 2. As such, the actuation mechanism 7 is provided between the outer part 24 and the inner part 25 of the housing 2.

The proximal section 22 of the housing further comprises a release valve 29. In the shown embodiment, the release valve 29 is provided in the inner part of the housing 25 and serves to release built up pressure in the discharge volume 26 as the moving plug 5 and/or soil enters the sampling device 1. The fluid (gaseous or fluid) will be released through the release valve 29 and into a pressure release space.

The drive ring 71 is coupled to the at least one extending actuation rod 73 and is further coupled to a drive sleeve 72, which is integrally formed with a cylindrical ring 74 provided at the top of the drive sleeve 72. In the embodiment shown in FIG. 5, the drive ring 71 is coupled to two longitudinally extending actuation rods 73, which are connected to the sampling unit 3. The movement of the actuation rods 73 induces a rotation of the sampling unit 3 between the open and closed positions.

The drive ring 71 is arranged to engage with a lower portion of the drive sleeve 72 such that a movement of the drive sleeve 72 will induce a movement of the drive ring 71. The actuation mechanism 7 further comprises a biasing element in the form of a helical spring 75, which is provided within the proximal section 22 of the housing, between the inner part 25 and the outer part 24 of the housing. It is noted that the biasing element may also comprise a disk spring, hydraulic spring, or the like.

The outer part 24 of the housing 2 comprises an inwardly directed flange 241, which is arranged to engage with the lower part 27 of the housing 2. The inwardly directed flange 241 also provides an upper biasing surface which engages with lower end of the helical spring 75. Similarly, the cylindrical ring 74 provided at the top of the drive sleeve 72 provides a bottom biasing surface which engages with the upper end of the helical spring 75. As such, the helical spring 75 is wedged between the bottom biasing surface of the cylindrical ring 74 and the upper biasing surface of the inwardly directed flange 241.

As the cylindrical ring 74 is driven in a downward direction, the drive ring 71 is also moved in a downward direction, together with the at least one extending actuation rod 73 as shown in FIG. 5. During this movement, the helical spring 75 is compressed and acts as a biasing element to push the cylindrical ring 74 back upwards after the pressure on the cylindrical ring 74 is alleviated. With that, the helical spring 75 pushes the cylindrical ring 74 and the drive ring 71 back upwards, and thereby retracts the at least one extending actuation rod 73. As such, the movement of the sampling unit 3 between the open and the closed position can be induced by providing a downward force to the cylindrical ring 74, for example with hydraulic fluid. To reverse the movement, the force to the cylindrical ring 74 is reduced, such that the helical spring 75 moves the longitudinally extending actuation rods 73 back upwards.

The actuation mechanism 7 in the shown embodiment further comprises an actuation pressure release unit 76 which is arranged to release the fluid (gaseous or liquid) surrounding the helical spring 75 between the outer part 24 of the housing 2 and the drive sleeve 72. As the cylindrical ring 74 is moved downwards and the spring 75 is compressed, the space in which the spring 75 is housed is reduced. To prevent an increase of pressure traversing the force provided to the cylindrical ring 74, the actuation pressure release unit 76 allows a fluid connection to a pressure release space 28 through a pressure release conduit 77. The pressure release conduit 77 is connected with the cylindrical ring 74, such that an actuation of the cylindrical ring will move the pressure release conduit 77, maintaining the fluid connection to the pressure release space.

To provide a driving force to the cylindrical ring 74, a hydraulic fluid may be applied through a hydraulic conduit 79. The hydraulic conduit 79 extends through a hydraulic seal 78 to a hydraulic drive space 70 defined by the top of the cylindrical ring 74 and the hydraulic seal 78. The pressure release conduit 77 similarly extends through the hydraulic seal 78. However, the pressure release conduit 77 also extends through the cylindrical ring 76. As such, while the hydraulic drive space 70 is pressured and increases in volume as the cylindrical ring 74 is pressed downwards, the fluid around the helical spring 75 can escape through the pressure release conduit 77 to the pressure release space 28.

In the shown example embodiment, the sampling device 1 further comprises a cross-over unit 8, arranged to couple the sampling device 1 to a drive system, which is arranged to drive the sampling unit into a target soil. The cross-over unit 8 according to the shown example embodiment houses the pressure release space and the hydraulic conduit. It may be coupled to known drive systems, such as cone penetration systems and the like.

Now referring to FIG. 2, a cross-sectional view of the sampling device according to one example embodiment of the invention in open position is shown. The features described in relation to FIG. 1 apply to FIG. 2 in similar fashion. The actuation mechanism 7 has been engaged such that the sampling unit 3 has been rotated into its open position. As a result, the first opening 31 faces towards the open end 23 of the distal section 21 of the housing 2. The second opening 32 faces upwards, towards the discharge volume 26 provided in the proximal section 22 of the housing 2 and delimited by the inner part 25 of the housing 2. As discussed in relation to FIG. 1, the container 6 comprises an open bottom 61 and an open top 62, which align with the first opening 31 and the second opening 32 of the sampling unit 3 in the open position.

The longitudinally extending actuation rods 73 have been driven downward by the application of pressure through the hydraulic conduit 79, through the hydraulic seal 78, into the hydraulic drive space 70. This has pressured the cylindrical ring 74 and the drive sleeve 72, against the pressure of the helical spring 75. The drive sleeve 72 is engaged with the drive ring 71, which is integrally formed with the longitudinally extending actuation rods 73 to actuate the sampling unit 3 to the open position.

In the shown embodiment, the moving plug 5 is driven upwards into the discharge volume 26 by the ingress of soil. The discharge volume 26 is larger than the moving plug 5, such that any soil under the sampling device 1 which may be slightly compressed, travels through the open end 23 of the distal section 21 of the housing 2, through the sampling chamber 4, into the discharge volume 26. The soil sample which then remains in the sampling chamber 4 is undisturbed.

Once the sample is retained in the sampling chamber 4, the pressure in the hydraulic drive space 70 is released, such that the spring 75 forces the drive sleeve 72 upwards, which pulls the drive ring 71 and the longitudinally extending actuation rods 73 in an upward direction such that the sampling unit 3 is rotated to a closed position, in which the first opening 31 and the second opening 32 are sealed by the container.

The sampling device 1 may then be retrieved from the soil and brought to the surface. To retrieve the sample, the cutting shoe 210 is disengaged from the lower part 27 of the housing 2 by a threaded connection. The container 6 with the sampling unit 3 can then be removed from the housing 2 of the sampling device.

Afterwards, a new container 6 with sampling unit 3 may be provided in the housing 2 of the sampling device 1, and the cutting shoe 210 may be screwed into place. A further sample may then be taken by the sampling device 1.

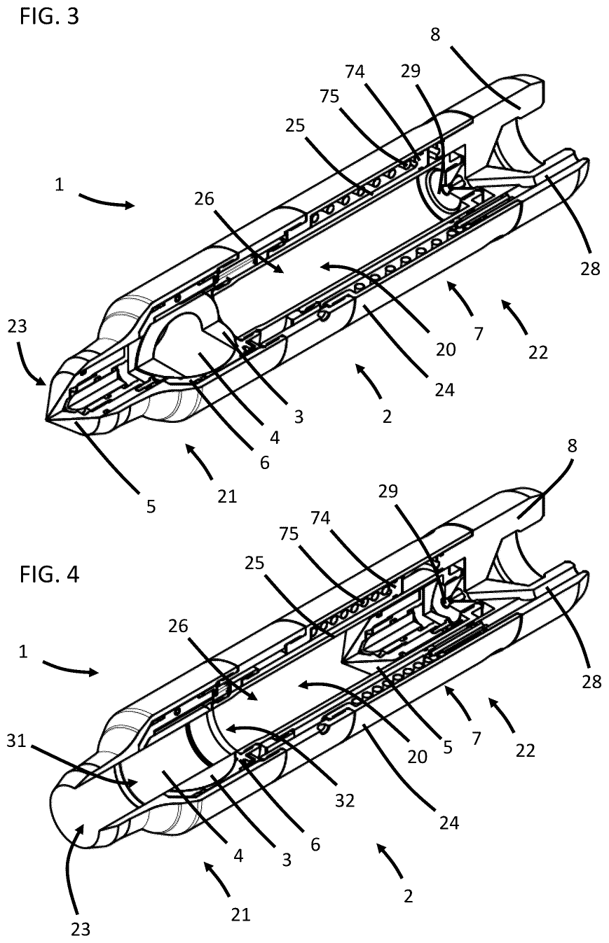

Now referring to FIG. 3 and FIG. 4, a three-dimensional cut-away view of the sampling device 1 according to one example embodiment of the invention is shown, in closed and open position, respectively. The shown example embodiment of the invention shows a sampling device 1 with a housing 2 having a distal section 21 and a proximal section 22. The sampling device 1 comprises a sampling unit 3, having a volumetrically calibrated sampling chamber 4. The sampling unit 3 is provided in a container, which is provided in the distal section 21 of the housing 2, more particularly, in the cutting shoe 210 of the distal section 21 of the housing 2.

The sampling device comprises a moving plug 5, which is arranged to travel through the sampling device 1 after the actuation mechanism 7 moves the sampling unit 3 from the closed position to the open position and the sampling device 1 is driven further into the soil. As shown in FIG. 4, the moving plug 5 is driven through the sampling device 1 to the discharge volume 26 of the housing 2. As the moving plug 5 is driven into the discharge volume, fluid previously present in the discharge volume 26 is forced out through the release valve 29 into the pressure release space 28. Similarly, as the space around the helical spring 75 is reduced, the excess fluid is also driven into the pressure release space.

Once a sample is in the sampling chamber 4, the actuation mechanism 7 moves the sampling unit 3 back into the closed position, and the sampling device 1 is retracted from the soil.

Now referring to FIG. 5, a blow-out view of the sampling device 1, showing the sampling unit 3, the container 6, and part of the actuation mechanism 7 according to an example embodiment of the invention is shown. The sampling unit 3 has a substantially spherical shape having flattened sides 33. The sampling unit 3 in the shown example embodiment shows two pins, arranged to extend into slots 731 provided in the longitudinally extending rods 73, which are coupled to the drive ring 71. The actuation rods 73 may be is coupled to the sampling unit 3 via the pins 34, which extend into the slots 731 of the rods 73. The pins 34 extend into the sampling unit 3, or are formed integrally with the sampling unit 3, in a position away from an axis of rotation of the sampling unit 3, such that an axial force exerted to the rods 73 creates a rotational moment on the sampling unit 3 around the axis of rotation to move between an open and a closed position.

The container 6 is vertically cut such that it comprises a first half 63 and a second half 64. The container 6 is provided around the sampling unit 3 such that the sampling unit 3, with the container 6, can be retrieved from the housing 2 as a single unit. The container 6 further comprises a compression ring 65, which can be provided around an upper flange 67 of the halves of the container to lock them together. Between the compression ring 65 and the flange 67, a sealing ring 66 is provided. The container 6 further comprises a locking ring 68, which can be provided around the container 6 halves 63, 64, in a groove 69 provided around the circumference of the container 6 to lock the halves 63, 64 together.

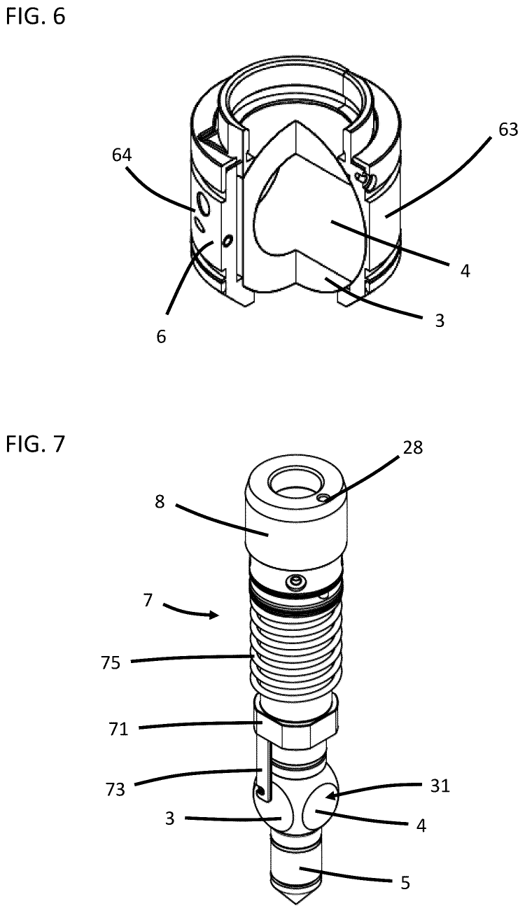

Now referring to FIG. 6, a three-dimensional cut-away view of the sampling device 1 showing the sampling unit 3, provided within the container 6, according to an example embodiment of the invention is shown. The container 6 comprises a first half 63 and a second half 64, which enclose around the sampling unit 3 such that the sampling chamber 4 is sealed by the container 6.

Now referring to FIG. 7, a three-dimensional view of the sampling device 1, showing the sampling unit 3, the moving plug 5, and at least a part of the actuation mechanism 7, according to an example embodiment of the invention is shown. The sampling device 1 of the shown example embodiment has a sampling unit 3, and a volumetrically calibrated sampling chamber 4 defining an opening 31 in the sampling unit 3.

The sampling device 1 further comprises a moving plug 5, arranged to move through the sampling chamber 4 if the actuation mechanism 7 rotates the sampling unit 3 into its open position and the sampling device 1 is driven further into the soil. The sampling device 1 according to the shown example embodiment further comprises a cross-over unit 8, arranged to be coupled to a drive system to drive the sampling device 1 into a target soil. The pressure release space 28 is provided in the cross-over unit and is arranged to release pressure from fluid around the spring 75 and from within the discharge volume 26.

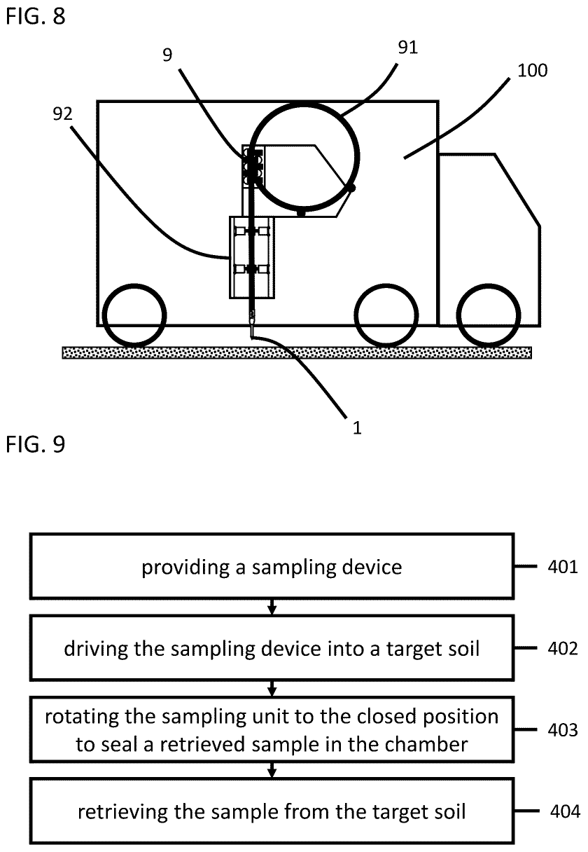

Now referring to FIG. 8, a side view of the sampling device 1 attached to a drive system 9 provided in a truck 100, according to an example embodiment of the invention is shown. The shown example embodiment shows a coiled drive system 9 having a coiled rod 91 provided in the truck 100, which is driven into the soil by the drive system 9, through a guide 92.

Now referring to FIG. 9, a schematic diagram showing a method of retrieving a soil sample to determine a void ratio of said soil sample according to an example embodiment of the invention is shown. The shown method comprises the steps of providing 401 a sampling device 1 according to any of the embodiment in this disclosure; driving 402 the sampling device 1 into a target soil to a pre-determined target depth; rotating 403 the sampling unit 3 from the open position into the closed position to seal a retrieved sample in the volumetrically calibrated sampling chamber 4; and retrieving 404 the sampling device 1 from the target soil.

The invention has been described by reference to certain embodiments discussed above. It will be recognized that these embodiments are susceptible to various modifications and alternative forms well known to those of skill in the art.

Further modifications in addition to those described above may be made to the structures and techniques described herein without departing from the spirit and scope of the invention. Accordingly, although specific embodiments have been described, these are examples only and are not limiting upon the scope of the invention.

1. A sampling device for retrieving a soil sample from a target soil to determine a void ratio of said soil sample, comprising:
a housing, arranged to be coupled to a drive system, the housing defining a distal section and a proximal section, the distal section of the housing having an open end arranged to receive soil as the sampling device is driven into the target soil;
a sampling unit provided in a space defined by the housing, the space extending into the housing from the open end of the distal section, the sampling unit comprising a volumetrically calibrated sampling chamber, the volumetrically calibrated sampling chamber extending at least partly through the sampling unit, such that the volumetrically calibrated sampling chamber defines at least one opening in the sampling unit,
wherein the sampling unit is rotatably connected to the housing such that it can rotate between:
a closed position wherein the at least one opening defined by the volumetrically calibrated sampling chamber faces away from the open end of the distal section of the housing, and wherein the at least one opening in the sampling unit is sealed, and
an open position wherein the at least one opening defined by the volumetrically calibrated sampling chamber faces the open end of the distal section of the housing; and
an actuation mechanism, arranged to rotate the sampling unit between the closed position and the open position.

2. The sampling device of claim 1, wherein the distal section of the housing is detachably connected to the proximal section.

3. The sampling device of claim 1, further comprising a container provided around the sampling unit, the container being provided in the housing.

4. The sampling device of claim 1, wherein the open end of the distal section of the housing axially aligns with the at least one opening in the sampling unit, defined by the volumetrically calibrated sampling chamber, when the sampling unit is in the open position.

5. The sampling device of claim 1, wherein the volumetrically calibrated sampling chamber defines a through-hole in the sampling unit.

6. The sampling device of claim 1, wherein the actuation mechanism comprises at least one longitudinally extending actuation rod, and wherein the sampling unit is coupled to the at least one longitudinally extending actuation rod, the at least one longitudinally extending actuation rod extending through the housing to the sampling unit.

7. The sampling device according to claim 6, wherein the at least one longitudinally extending actuation rod is coupled to the sampling unit via at least one pin, the at least one pin extending into the at least one longitudinally extending actuation rod and into the sampling unit in a position away from an axis of rotation of the sampling unit, such that an axial force exerted to the at least one longitudinally extending actuation rod create a rotational moment on the sampling unit around the axis of rotation.

8. The sampling device of claim 1, wherein the actuation mechanism comprises a hydraulic drive system, such that the sampling unit may be moved between the open position and the closed position by the increase or decrease of a hydraulic pressure.

9. The sampling device of claim 1, wherein the actuation mechanism comprises a cylindrical piston provided within the proximal section of the housing, the cylindrical piston being coupled to at least one actuation rod to drive rotation of the sampling unit.

10. The sampling device of claim 1, wherein the actuation mechanism comprises a biasing element to bias the sampling unit in the closed position or in the open position.

11. The sampling device of claim 10, wherein the biasing element is a helical spring, arranged within the proximal section of the housing, and wherein the actuation mechanism comprises a cylindrical piston provided within the proximal section of the housing, wherein the biasing element is arranged to bias the cylindrical piston away from the sampling unit.

12. A method of retrieving a soil sample to determine a void ratio of said soil sample, comprising:
providing a sampling device;
driving the sampling device into a target soil to a pre-determined target depth;
rotating a sampling unit from an open position into a closed position to seal a retrieved sample in the-a volumetrically calibrated sampling chamber; and
retrieving the sampling device from the target soil.

13. The method according to claim 12, wherein the sampling unit is in the closed position as the sampling device is driven into the target soil, and wherein the method further comprises:
once the sampling device is at the pre-determined target depth, rotating the sampling unit from the closed position into the open position; and
driving the sampling device further into the target soil for a pre-determined distance, prior to rotating the sampling unit back into the closed position.

14. A kit of parts for assembling a sampling device, the kit comprising:
a housing, arranged to be coupled to a drive system, the housing defining a distal section and a proximal section, the distal section of the housing having an open end arranged to receive soil as the sampling device is driven into the soil;
a sampling unit arranged to be provided in a space defined by the housing, the space extending into the housing from the open end of the distal section, the sampling unit comprising a volumetrically calibrated sampling chamber, the volumetrically calibrated sampling chamber extending at least partly through the sampling unit, such that the volumetrically calibrated sampling chamber defines at least one opening in the sampling unit, wherein the sampling unit is arranged to be rotatably connected to the housing such that it can rotate between:
a closed position wherein an opening defined by the volumetrically calibrated sampling chamber faces away from the open end of the distal section of the housing, and wherein the at least one opening in the sampling unit is sealed, and
an open position wherein the opening defined by the volumetrically calibrated sampling chamber faces the open end of the distal section of the housing; and
an actuation mechanism, arranged to rotate the sampling unit between the closed position and the open position.

15. (canceled)

16. The kit of claim 14, wherein the distal section of the housing is detachably connected to the proximal section.

17. The kit of claim 14, further comprising a container provided around the sampling unit, the container being provided in the housing.

18. The kit of claim 14, wherein the open end of the distal section of the housing axially aligns with the at least one opening in the sampling unit, defined by the volumetrically calibrated sampling chamber, when the sampling unit is in the open position.

19. The kit of claim 14, wherein the volumetrically calibrated sampling chamber defines a through-hole in the sampling unit.

20. The kit of claim 14, wherein the actuation mechanism comprises at least one longitudinally extending actuation rod, and wherein the sampling unit is coupled to the at least one longitudinally extending actuation rod, the at least one longitudinally extending actuation rod extending through the housing to the sampling unit.

21. The kit according to claim 20, wherein the at least one longitudinally extending actuation rod is coupled to the sampling unit via at least one pin, the at least one pin extending into the at least one longitudinally extending actuation rod and into the sampling unit in a position away from an axis of rotation of the sampling unit, such that an axial force exerted to the at least one longitudinally extending actuation rod create a rotational moment on the sampling unit around the axis of rotation.
